(19) United States
(12) Patent Application Publication
(10) Pub. No.: US 2017/0035990 A1
(43) Pub. Date: Feb. 9, 2017

(54) ENDOLUMINAL FLUID DELIVERY DEVICE AND METHOD

(71) Applicant: Kevin Swift, Brighton, MA (US)

(72) Inventor: Kevin Swift, Brighton, MA (US)

(21) Appl. No.: 15/228,751

(22) Filed: Aug. 4, 2016

Related U.S. Application Data
(60) Provisional application No. 62/200,788, filed on Aug. 4, 2015.

(51) Int. Cl. A61M 25/00; A61M 25/01; A61M 25/06

(52) U.S. Cl. CPC A61M 25/0084; A61M 25/0606; A61M 25/0169; A61M 25/0071; A61M 2025/009

(57) ABSTRACT

A device and method for delivering a drug from inside a body lumen to tissue surrounding the body lumen. An endoluminal drug delivery device is connectable to a drug source and includes a dual-lumen catheter, including a treatment device lumen for housing a guidewire and/or a treatment device and a needle lumen for housing a retractable needle. The guidewire exits the catheter through an opening at the distal end, and the needle exits the catheter through an exit port in the outer wall of the catheter.

### FIELD OF THE INVENTION

The present invention relates generally to devices and methods for delivering medicinal substances to an area surrounding a hollow tubular structure. More specifically, the invention relates to an improved device and method for delivering fluid to an extravascular space using a needle which pierces through the tubular structure.

### CROSS-REFERENCE TO RELATED APPLICATION

This application claims priority to U.S. Provisional Application No. 62/200,788, filed Aug. 4, 2015, which is incorporated in its entirety herein.

### BACKGROUND OF THE INVENTION

Normally, valves in veins keep blood moving toward the heart and prevent backflow. In patients with varicose veins, the valves do not function properly, so blood flows back toward the patient's extremities and pools in the veins. This can lead to skin necrosis. Laser or RF ablation treatment creates hypercoagulability to close the abnormal vein, which is subsequently absorbed by the body. The current commonly-used method of delivering numbing medication to the treatment area prior to the ablation procedure requires multiple injections in the patient's thigh, each through the skin. Disadvantages of this method can include pain for the patient and inefficiency.

Medical professionals commonly use catheters for gaining access to an area within the body. A common procedure where the catheter is positioned in an area in order to receive treatment is varicose vein treatment. Treatment of varicose veins is usually performed using some form of thermal energy emitting device that is placed in the vein with a catheter and guidewire. Energy types may include laser, radiofrequency, microwave and cryoablation. Before treatment of the vein using a thermal device, tumescent, or some other form of anesthetic is used to numb the area surrounding the vein in order to reduce discomfort and accidental damage to the area outside the vein. The process of delivering tumescent is rather tedious and is far from an exact process. The current method of delivering tumescent uses repeated injections from a short needle. Tumescent injections, typically a lidocaine/saline, are administered along the entire length of the targeted vein segment using ultrasonic guidance. The tumescent injections perform several functions. The anesthesia inhibits pain caused from the application of thermal energy to the vein. The tumescent injection also provides a barrier between the vessel and the adjacent tissue and nerve structures, which restricts the damage to within the vessel and prevents non-target tissue damage. The injection of fluid into the peri-venous sheath also causes the targeted vein segment to become compressed, resulting in a smaller vein diameter. The smaller diameter vein is desirable, particularly for non-thermal treatments such as sclerotherapy, glue or other chemical agent applications, which the agent to contact the vessel wall for effective treatment. Once the needle is properly placed, the fluid is administered by syringe or pump. The injections are repeated along the entire length of the treatment area.

The process of delivering tumescent fluid prior to a vein ablation procedure is time consuming and cumbersome. The delivery of tumescent fluid is typically the longest segment of the vein ablation procedure and is often the cause of procedural complications due to the difficulty of precisely locating the needle in the perivenous sheath for multiple sticks. It is often difficult for the physician to single-handedly administer the fluid because he/she must hold the ultrasound probe with one hand, position the needle with the other while deploying the plunger if a syringe is used. The physician may advance the needle too far (into the vein or through the far wall of the vein) or not far enough resulting in incomplete fluid delivery and the resultant procedural complications.

U.S. Pat. No. 8,852,165, which is herein incorporated by reference, attempts to address these problems by providing an endoluminal drug delivery device which includes a dual lumen catheter which houses a guidewire and a needle connectable to a drug source. After the device is placed within the vein, the needle is advanced exiting out of a side hole in the catheter wall into the fascial space where drug is delivered. The needle is then retracted and re-advanced through a more proximal side hole in the catheter wall. Although addressing the problem of multiple injections through a patient's skin, the '165 device fails to solve other problems associated with the varicose vein treatment procedure.

The invention described herein addresses a problem of the device described in the '165 patent. As described below this invention includes maintaining the needle tip in the same axial orientation relative to the catheter wall during the needle advancement and retraction steps. The needle tip of the '165 patent may become misaligned within the catheter lumen as it is advanced and retracted. The anatomy of the vein, which may be tortuous, as well as the tendency of the vein to spasm during a procedure, may also cause misalignment of the needle tip within the catheter. Once misaligned, the user may not be able to manipulate the needle tip back in axial alignment with the designated exit hole, even with ultrasound guidance.

Another difficulty with the device described in the '165 patent is the need for multiple device component exchanges. Once the tumescent delivery step has been completed, the user must remove the endoluminal drug delivery device and replace it with a procedure sheath. The laser fiber or other thermal device is then inserted into the procedure sheath for the treatment step. Multiple device component exchanges lead to a longer procedure and potential for additional complications. An advantage of one embodiment of this invention is to provide the user with the ability easily and quickly gain access with the ablation device using a single device. This invention reduces procedure time and also reduces the possibility for additional human error by removing the step of having to replace the drug delivery device with a procedure sheath.

Yet another drawback of the device and method described in the '165 patent involves the unintended flow of blood, saline and other treatment fluids through side exit holes in the catheter shaft. A typical step in the preparation procedure is to flush the catheter lumen with saline to remove any debris and air which may be present in the passage. The presence of side holes prevents the user from being able to complete this step because saline will flow out of the side holes instead of flowing through the entire catheter and exiting out of the end hole. During the procedure as the device is withdrawn, blood enters through the side holes into the catheter lumen. Extension of blood within the lumen may result in catheter occlusion. In addition, withdrawal of the device to the point where a side hole is exposed will result in blood spillage onto the patient and/or treatment area.

Accordingly, there is a need for an improved endoluminal drug delivery device which guarantees needle alignment throughout the procedure, minimizes the number of device component exchanges and ensures that procedural and patient fluids do not jeopardize the procedure by exiting out of the side holes in the device. The method of using the endoluminal drug delivery device should also minimize the number of procedural steps required to treat the patient.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1 is a longitudinal plan view of one embodiment of the endoluminal drug delivery and treatment device having a dual lumen configuration.

FIG. 2 is a cross-sectional view of the endoluminal device of FIG. 1 taken along line A-A depicting the needle in a retracted position.

FIG. 3A is a cross-sectional view of the endoluminal device of FIG. 1 taken along line B-B depicting the needle in a deployed position.

FIG. 3B is a partial isometric view of the endoluminal device of FIG. 1 with the needle in a deployed position and depicting the guidewire exiting from the distal end of the device.

FIG. 4 is a partial plan view of the distal section of the needle component.

FIG. 5 is a partial plan view of the intermediate section of the needle component.

FIG. 6 is a partial cross-sectional view of the endoluminal device illustrating the needle in a deployed position.

FIG. 7 is a partial cross-sectional view of the endoluminal device illustrating the needle in a retracted position.

FIG. 8 is a partial cross-sectional view of the distal end of the endoluminal device illustrating the needle starting to deploy through a side hole exit.

FIG. 9 is a partial longitudinal plan view of Embodiment 2 of the endoluminal device having a single lumen configuration.

FIG. 10A is an isometric plan view of the endoluminal device of FIG. 8 illustrating hub and handle components.

FIG. 10B is an enlarged isometric partial view of the distal end section of the endoluminal device of FIG. 8.

FIG. 11A is a partial longitudinal cross-sectional view of the endoluminal device of FIG. 9 showing inner catheter/outer sheath elements with the needle extending from the distal end of the inner catheter.

FIG. 11B is a cross-sectional view of the endoluminal device of FIG. 11A taken along lines A-A and B-B.

FIG. 12A is a longitudinal plan view of Embodiment 4 of the endoluminal device having a dual lumen configuration.

FIG. 12B is a longitudinal plan view of Embodiment 4, with a RF probe/optical fiber attached.

FIG. 13A-A is a cross-sectional view of the endoluminal device of FIG. 12A taken along lines A-A.

FIG. 13B-B is a cross-sectional view of the endoluminal device of FIG. 12B taken along lines B-B.

FIG. 14A is a partial longitudinal plan view of the distal end of the endoluminal device of Embodiment 4 with the needle in the deployed position.

FIG. 14B is a partial longitudinal plan view of the distal end of the endoluminal device of FIG. 12B with the needle in the retracted position.

FIG. 15A is a flow chart outlining the steps needed to insert and use another embodiment of the endoluminal device.

FIG. 15B is a flow chart outlining the steps needed to insert and use another embodiment of the endoluminal device.

FIG. 15C is a flow chart outlining the steps needed to insert and use another embodiment of the endoluminal device.

FIG. 16 is a plan view of the distal end of another embodiment of the endoluminal device.

### DETAILED DESCRIPTION OF THE INVENTION

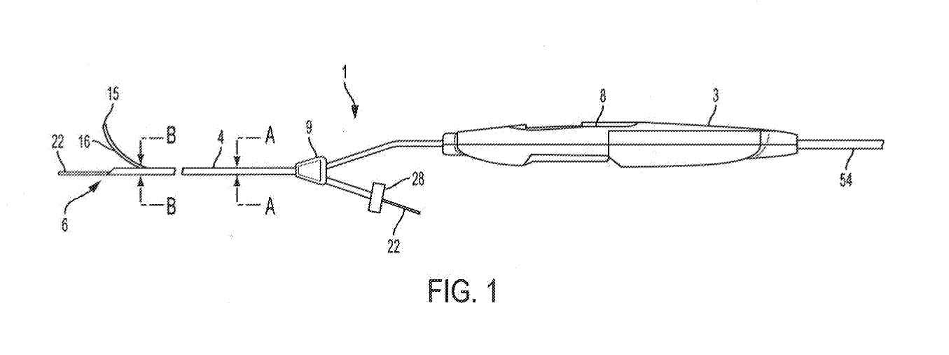

One embodiment of the present invention is shown in FIG. 1 through FIG. 8. The endoluminal device 1 is comprised of a handle 3 connectable to a tumescent fluid source (not shown), a catheter shaft 4, having either a double lumen configuration or single lumen configuration (not shown), a hub 9 and a needle 5. The handle may include a thumb slide 8 or other mechanism for deployment and retraction of the distal end section of a needle 5 from the catheter shaft 4 through a side exit hole 10. The elongated needle 5 is disposed within catheter shaft 4. The needle 5 includes a through lumen for fluid delivery, such as tumescent or other anesthetic, and may have a curved profile at the distal end section to facilitate positioning and advancing of the needle tip 15 through the vein wall into the perivenous space when the needle 5 is advanced through side exit hole 10. A hub 9, positioned on the catheter shaft 4 distal of the handle 3, provides an access point for inserting a guidewire 22 into the catheter shaft.

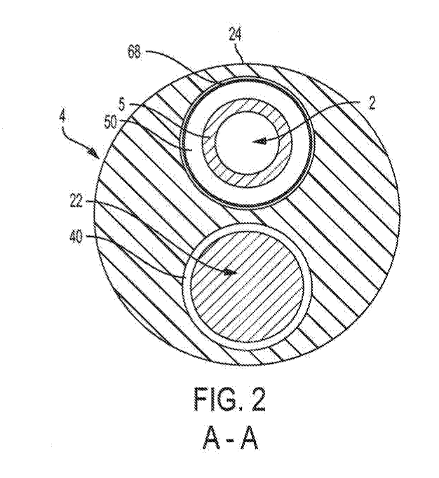

The catheter shaft 4 may range from 4F to 1° F. in size and may be comprised of a urethane or PEBAX extruded material or of such construction as is disclosed in U.S. Pat. No. 7,458,967 to Kabnick, et. al. In one embodiment, the shaft 4 is a dual-lumen configuration as shown in the cross-sectional view of FIG. 2.

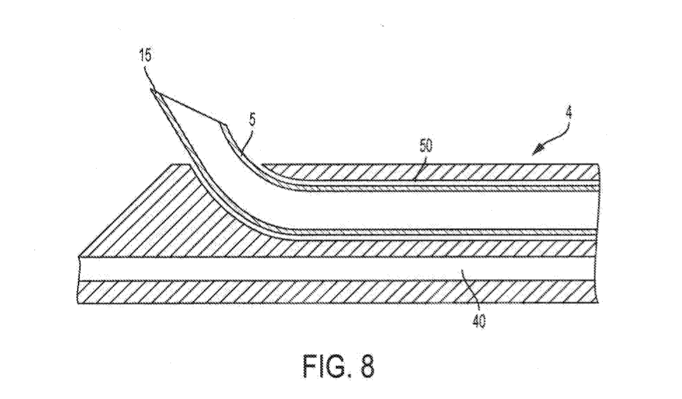

Bottom guidewire lumen 40 is designed to accept a standard guidewire 22 having an outer diameter of 0.035″ although other sizes are within the scope of this invention. Needle lumen 50 is designed to accept a hollow needle assembly 5. The guidewire lumen 40 extends to and through the distal tip 6 of the catheter 4. The needle lumen 50 extends from the proximal end of the shaft 4 and terminates a selected distance distal of the side exit hole 10, as shown in FIG. 8. The catheter shaft 4 may include an extruded strip 24 extending the length of the shaft 4 over the needle lumen 50 to assist the user in identifying the relative positions of the lumens so that the device may be maintained in the correct orientation in the vessel.

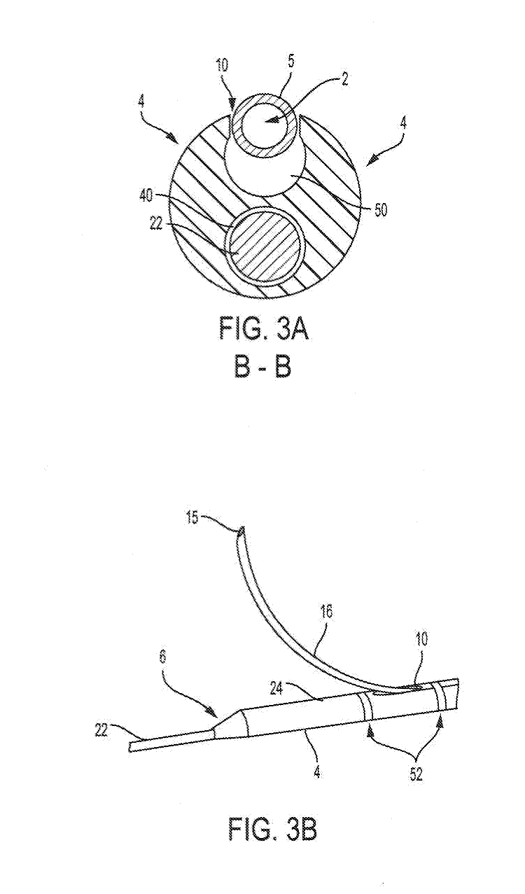

Adjacent the distal end 6 of the catheter 4 is a side exit hole 10 formed in the catheter shaft wall as shown in FIGS. 2A and 3A. The side exit hole 10, which is in communication with the needle lumen 50, forms an exit pathway for the distal curved section 16 of the needle 5 when the thumb slide 8 is activated causing the needle assembly to advance. Distal curve section 16 may be between 0.5 to 2.0 cm in length. The side exit hole 10 walls may have a chamfered or skived profile to facilitate advancement of the bevel needle tip 15 through hole 10. The terminal tip 15 of the needle lumen 50 may also be of an beveled or chamfered profile to assist in needle advancement.

Markers 52, such as an echogenic markers, may be positioned adjacent the side exit hole 10 to provide the user with enhanced ultrasound visualization of the side exit hole 10 prior to needle 5 deployment. The bands 52 assist the physician in precisely locating the exit hole 10 under ultrasound guidance. The marker bands 52 will provide additional visibility under ultrasound either by having a brighter reflection or showing a different contour than the outer body of the catheter 4. By knowing the position of the marker band 52 relative to the hole 10, a physician can then position the needle tip 15 at a precise location and obtain greater accuracy in guiding the needle out of the catheter 4. The marker bands 52 may be made of metal other polymer or ceramic echogenic materials. A single marker band 52 may designate the location of the exit side hole or marker bands may be positioned on either side of the exit hole.

The needle lumen 50, in which the needle 5 resides is in fluid communication with a channel in the device handle 3. This channel, in turn, is connected to the tumsecent line 54 which extends proximally from the handle 3 and connects to a tumsecent fluid source (not shown). In a typical varicose vein treatment procedure, the fluid source is connected to a pump which is activated by the clinician via a foot pedal. In addition, the fluid source could be connected to a hand syringe for fluid delivery. When depressed, the foot pedal causes the pump to activate causing fluid to flow from the fluid source through the tumsecent line 54, through the handle 3 channel and finally through the needle 5 lumen where it exits through the needle tip 15.

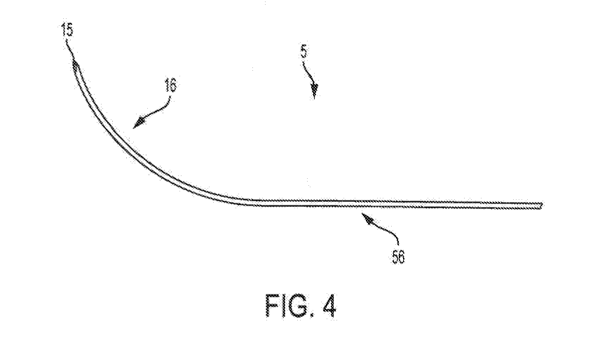

The needle assembly, which provides the fluid pathway for delivery of the tumescent anesthesia or other fluid to the perivenous space, is comprised of a hollow needle 5 have a straight shaft portion 56, a curved portion 16 and a bevel needle tip 15 as shown in FIG. 4. In one embodiment the needle assembly has longitudinal through lumen 2 having an inner diameter of 0.020″. The needle assembly may have an outer diameter of 0.028″, although other sizes are within the scope of this invention.

The needle 5 may be of a single stiffness for the entire length or of a variable stiffness. In one configuration the needle assembly is comprised of a nitinol hollow needle 5 formed with the desired curve portion 16 and bevel tip 15 for puncturing through the vessel wall. In another less costly configuration, the needle assembly is comprised of a polyamide or stainless steel straight shaft portion 56A which is attached to a nitinol shaft having a short length of un-curved shaft in addition to the curved portion 16. The polyimide is a cost-effective material that can be easily bonded to the stainless steel and nitinol metal components and has an adequate stiffness to advance/retract the needle 5 when contained within the catheter shaft. Being more flexible than an only stainless steel or nitinol needle, this embodiment may have enhanced tracking capabilities through more tortuous venous anatomies. In addition, the polyamide and/or stainless steel straight shaft portion provides a stiffer material that will not kink or deform within the handle 3, where there is an open space between the handle 3 and the thumb slide 8 to which the needle 5 is connected. A stiffer needle material also allows the user better control when actuating the thumb slide 8. In another configuration, the proximal section of the needle 56A may be of the highest stiffness, stainless steel for example, the intermediate section 56B may be of lower stiffness and the distal curved portion 16 may be of an intermediate stiffness. In another configuration, the needle assembly could be made entirely of stainless steel, which would be more cost-effective than using a needle assembly using all nitinol and/or a needle assembly with polyamide/stainless steel and nitinol. As stated above, a stainless steel needle assembly will provide for a stiffer assembly, allowing allows for better control for the user when actuating the thumb slide 8.

Referring to FIG. 4, the distal end section 16 of the needle 5 may be media-blasted to create a rougher outer surface that will increase visibility of the curved section 16 of the needle 5. The super-elastic qualities of nitinol distal end section 16 will allow the needle 5 to be straightened out when confined within the ED device 1 catheter shaft 4. As the needle tip 15 is advanced out of the side hole 10, the unconstrained needle end section 16 will return to its pre-formed curved shape causing the distal bevel tip 15 to puncture the vessel wall and advance into the perivenous space for fluid delivery.

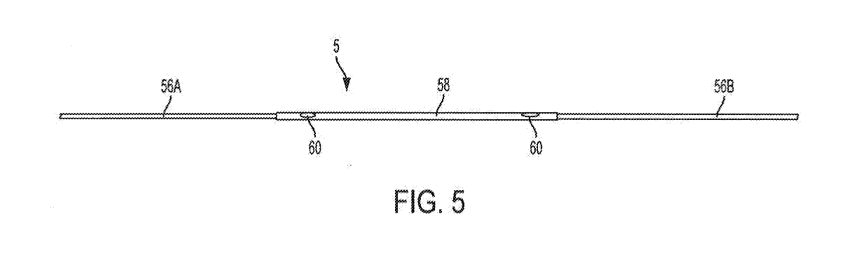

With the stainless steel/nitinol assembly, a stainless steel over sleeve 58 may be positioned over the abutting ends of the two needle components 56A and 56B as shown in FIG. 5. Openings 60 formed in the over sleeve may be used to apply an adherent such as glue to the needle components and the over sleeve 58. Optionally, glue may be applied to both ends of the over sleeve joints to form a fluid seal. The over sleeve 58 ensures a stable and reliable connection without the need for welding.

In another embodiment, the needle 5 could be made of a shape memory alloy, such as nitinol or a combination of a nitinol tip and a stiffer shaft portion, such as stainless steel or a polyamide material. Additionally, the needle can be made entirely of stainless steel, which would be more cost-effective than using a needle using all nitinol and/or a needle with polyamide/stainless steel and nitinol. As stated above, a stainless steel needle assembly will provide for a stiffer assembly, allowing allows for better control for the user.

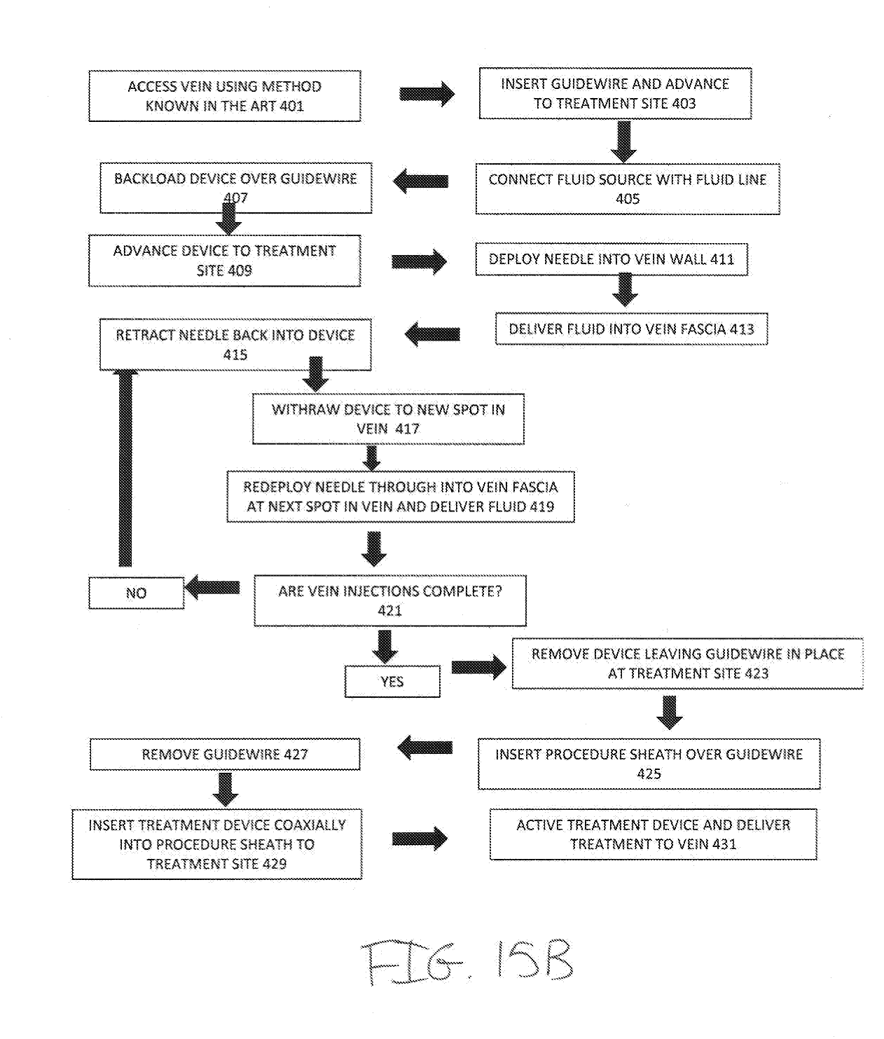

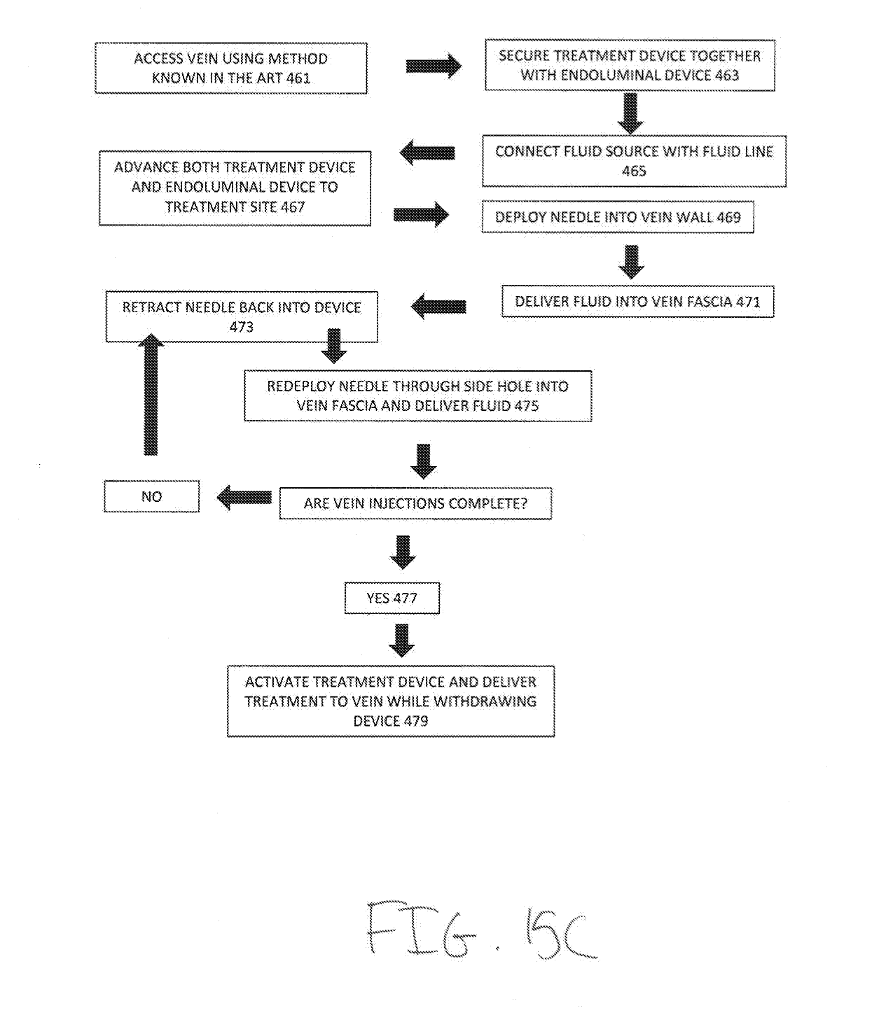

The method of using one embodiment of the dual lumen device 1 will now be described, as shown in FIG. 15A. The target vein is accessed using standard technique 401 after which a procedural guidewire 22 is inserted 403 and advanced to the desired location. The device 1 is connected to a fluid source by attaching the tumescent line 54 of the device 1 to the fluid source connector 405. With the needle 5 in a retracted position within the catheter shaft 4, device 1 is then inserted into the vessel by back loading 407 the guidewire 22 into the catheter guidewire lumen 40 and advancing until it extends proximally through a slit valve 28 and the hub 9.

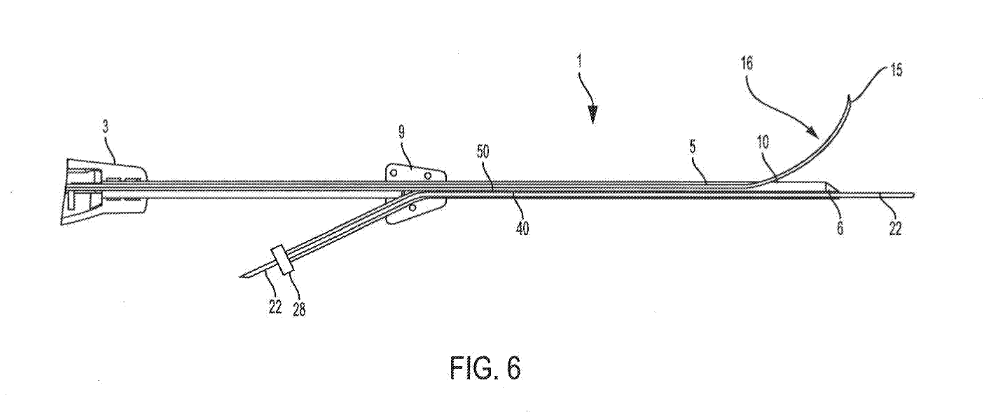

After advancing the device 1 through the vessel to the target location 409, the needle 5 may be deployed 411 by advancing the thumb slide 8. Advancement causes the needle distal end section 16 to exit through the side exit hole 10 and pierce through the vessel wall as shown in FIG. 6.

The thumb slide 8 may be used to regulate the extent of needle tip 15 advancement from the catheter shaft 4. The physician may utilize deployment length indicators on the handle 3 to limit the length of the needle 5 penetrating into the perivenous space. The needle throw may be between 0.5-2.0 cm. The physician may adjust the deployment length so that the needle 5 does not over-advance and enter surrounding untargeted tissue which would result in patient complications including procedure pain and swelling. Ultrasound visualization of the side hole 10, needle distal tip section 16 and perivenous sheath will assist the physician in determining optimal deployment length. Allowing the physician to set a needle deployment distance decreases complications resulting from a needle tip 15 being mispositioned relative to the target location.

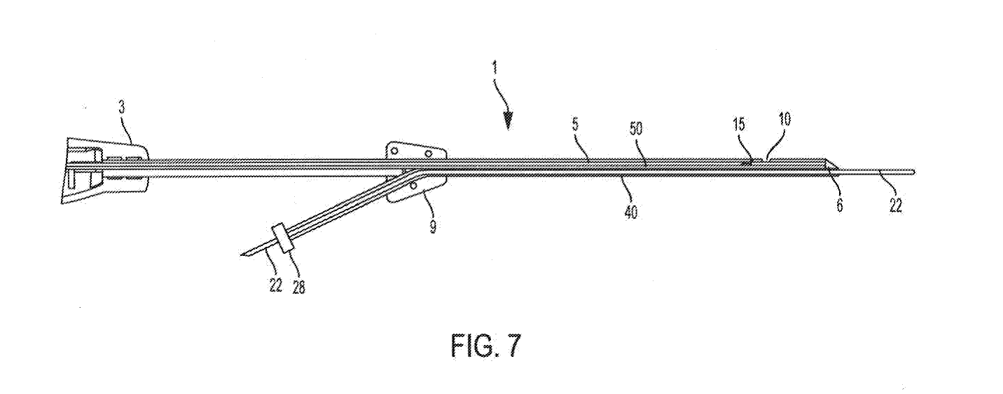

Once the needle tip 15 is in the perivenous sheath, the user injects tumescent fluid into the space 413 by depressing the pump foot pedal or by manually injecting using a syringe or other device. Once sufficient fluid has been delivered to the target location, the user retracts the thumb slide 8 to withdraw 415 the needle 5 from the perivenous space until the needle tip 15 is again positioned within the needle lumen 50 as shown in FIG. 7.

The user then withdraws the device 1 to a new position within the vein 417 after which the tumescent fluid is again delivered 419 using the steps described in the previous paragraph. The injection process is repeated at different locations along the vein until the entire length of the target vein has been treated 421. The user then removes the device 1 leaving the guidewire 22 in position within the vein 423. The vein is now ready for treatment using a laser fiber, or other treatment device. As described in U.S. Pat. No. 7,458,967, a procedure sheath is advanced over the guidewire to the target location 423. After removing 427 the guidewire 22, an optical fiber or other thermal delivery device in inserted into the sheath and advanced until it is positioned at the desired treatment location 429, typical 1-2 cm proximally of the sapheno-femoral junction if the great saphenous vein is being treated. Thermal energy, such as laser energy or RF energy, is applied along the entire length of the vein by withdrawing the sheath/fiber assembly at a designated withdrawal rate until the entire vein has been treated 431.

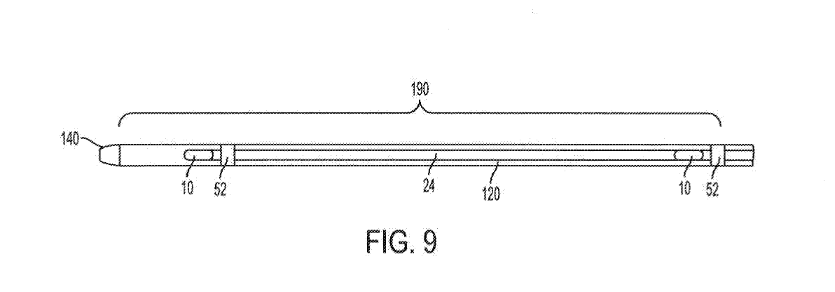

Another embodiment of the present invention is shown in FIG. 9 through FIG. 11B. The single lumen endoluminal device 100 includes of inner needle catheter 110, and outer catheter sheath 120. Within the lumen of the outer sheath 120 is positioned a single lumen 160 shaft extending between hub assembly 130 and terminates a selected distance distal to the distal tapered tip 140. The shaft size of the outer sheath 120 can range from 4-8F and with a lumen 160 size designed to accept a standard 0.035″ guidewire, although other sizes are within the scope of the invention. The single lumen design has the advantage of have an overall lower outer profile than prior art dual lumen design. The outer sheath 110 serves as a conduit for the inner needle catheter 110 which is inserted into the outer sheath 120 once the outer sheath has been placed in the vessel as will described in more detail below.

The outer sheath 120 includes multiple side exit holes 10 with marker bands 52 to assist in locating the holes during treatment. Holes 10 may be created using a drilling, punch or skiving process. In one embodiment, 5-15 holes are fixed at 5 cm intervals along the outer sheath 110.

The shaft 110 may have a thin outer layer 190 extending from the catheter tip 140 to proximal of the proximal most exit hole as shown in FIG. 9. The layer 190 may be comprised of heat shrink tubing or other similar material that can be applied to the outer sheath 120 after the marker bands 52 have been embedded or otherwise secured to the catheter shaft. The shrink tubing 190 is preferably transparent so holes 10 are visible to the user. The heat shrink layer 190 serves several purposes. The layer provides a tight seal over the exit holes 10 to prevent any blood present within or entering into the device through the side holes 10 before and during the procedure. Holes 10 become progressively exposed to procedure room atmosphere as the outer sheath 120 is retracted from the patient. The heat shrink material is thin and easily pierced by the needle tip 15. Since puncture holes left in layer 190 by the needle tip 15 are much smaller than the actual side hole 10, blood flow into and out of the catheter shaft during the procedure will be significantly reduced. Those side holes 10 which will not be used in the procedure remained covered thus preventing unnecessary blood loss and spillage.

The outer layer 190 also facilitates the application of a hydrophilic coating if desired. Because the heat shrink 190 covers the side exit holes 10, the hydrophilic coating may be applied without risking migration of the coating through the holes 10 in the outer sheath 160 and inner catheter lumen 150. During the hydrophilic curing process, any coating found on the inside of the device lumens could result in partial or complete occlusion of the lumens. With the added outer layer of shrink wrap, this problem is eliminated because the coating is blocked from entering the device interior through the side holes 10.

Another advantage of the outer layer 190 design in that it allows the physician to flush the device without risk of having saline spill through the side holes 10 onto unintended surfaces. Physicians typically flush the catheter devices to remove any debris and air from the inner lumen of the catheters and sheath. The outer layer 190 covers the holes 170 and so prevents saline loss through the side wall of the sheath. In addition the outer layer 190 allows for the saline to travel fully down the sheath to dispel air and debris along the entire length of the device 1.

In an alternative embodiment, the side holes 10 may be replaced with pressure responsive slits as described in U.S. Pat. Nos. 5,250,034 and 5,267,979, which are incorporated herein by reference. As described in these patents, the pressure responsive exit slits permit fluid and other material to exit from the catheter lumen in response to a pressure over a first pre-determine level while preventing material from entering into the catheter lumen at a pressure less than a second pre-determined level. The slits would eliminate the need for the outer layer 190. Undesired blood flow into the catheter lumen would not occur while the slits are closed. The slits would remain closed unless opened by the pressure exerted by the needle tip 15 as it advances through the slit and into the perivenous tissue. Retraction of the needle tip 15 through the slit would cause the slit to reclose, preventing any blood inflow. The normally closed slits would also allow the application of a hydrophilic or other coating without unintentional migration of the coating material into the catheter lumen through the slits. The slits would not open while flushing of the catheter, which is typically done under low pressure conditions, thus allowing fluid to flow through the entire lumen and out the end hole of the catheter without unintended saline spills through side holes.

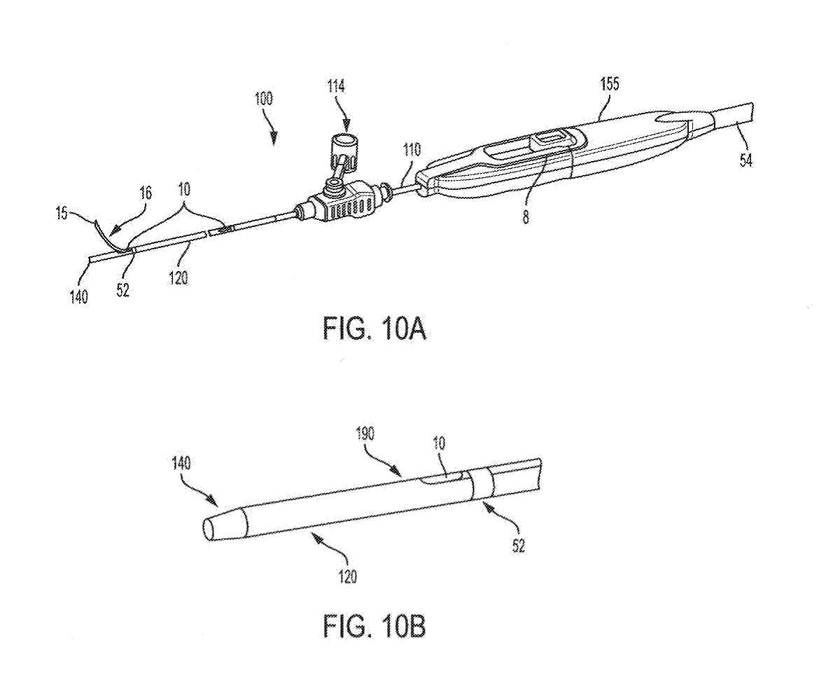

The inner needle catheter 110 is comprised of a single lumen needle catheter shaft 153 terminating in a distal tip 152 at one end and a handle 155 connectable to a tumescent fluid source (not shown) at the other end. An elongated needle 5 is disposed within catheter shaft 153 and includes a connection to the thumb slide 8 within the handle 155 as shown in FIG. 10A. The needle 5 includes a through lumen 2 for tumescent fluid delivery and may have a curved profile at the distal end section 16 to facilitate positioning and advancing of the needle tip 15 at an angle through the vein wall into the perivenous space as previously described.

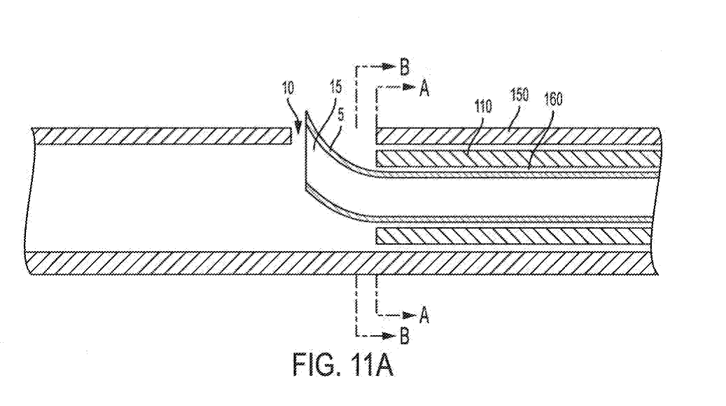

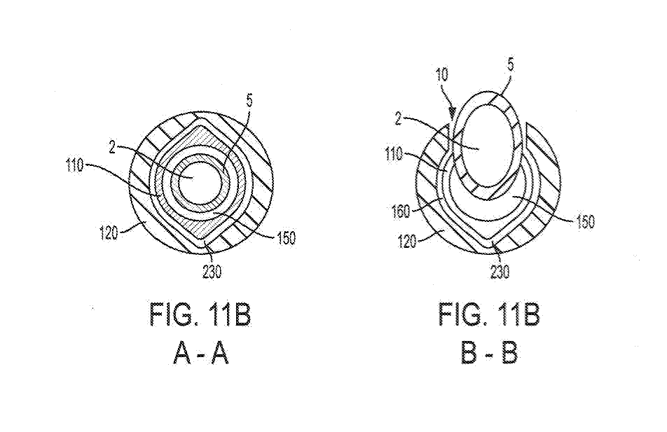

The outer catheter sheath 120 and inner needle catheter 110 have corresponding keyed geometries which prevent rotational movement between the two catheters when the inner needle catheter 110 is inserted into the outer catheter sheath 120. The keyed geometries of the two catheters can take many forms, including, but not limited to two key hole profiles, a single key hole profile at the top of the inner and outer catheters with a rounded profile at the bottom, a squared-off keyed profile at the top of the inner and outer catheters with a rounded profile at the bottom, and a rounded keyed profile at the top of the inner and outer catheters with a rounded profile at the bottom. As shown below in FIG. 10A, the inner catheter has a non-circular cross-sectional shape that includes two projecting key points 200. The outer sheath 120 has a corresponding inner wall geometry which includes two key hole 210 profiles where the sheath wall is of a reduced cross-sectional dimension, as shown in FIG. 10B. The projecting key points 200 of the inner needle catheter 110 are designed to align with the key hole 210 profiles of the outer sheath 120 when the inner needle catheter 110 is inserted into the outer sheath 120, as shown in FIG. 11A-A.

This uniquely shaped “keyed” geometry between the inner diameter of the outer sheath 120 and the outer diameter of the inner needle catheter 110 ensures that the inner catheter which houses needle 5 is aligned in one of two orientations (either zero or 180 degrees) when inserted into the outer sheath 120. The frictional interface between the two outer surface of key points 200 and inner surface of key holes 210 prevents any rotation of the inner catheter 110 relative to the outer sheath 120 when inserting the catheter 110 into the sheath 120 and during the actual procedure.

The alignment of inner catheter 110 is critical because the user needs to know the location and orientation of the needle tip 15 as well as ensuring that the needle 5 remains oriented correctly relative to the side exit holes 10 of the sheath. The needle is bonded to the handle 155 of the inner catheter 110 so that the needle tip 15 is oriented towards a designated key point 200 of the inner catheter 110. The needle tip 15 remains in a constrained, relatively straight position within the inner catheter 110 until deployed by actuating the thumb slide 8. A stainless steel insert 68, as shown in FIG. 2A, may be added to the distal section lumen 150 of the inner catheter 110 to prevent the catheter from taking on a “hockey stick” curve corresponding to the needle curve. It also prevents the needle tip 15 from embedding into the catheter wall while constrained.

The inner needle catheter 110 is inserted into to the sheath 120 such that the key points 200 become aligned within the designated key holes 200. The longitudinal stripe 24 on the outer wall of the outer sheath 110 provides an alignment indicator for the user to follow during insertion of inner needle catheter 110 into outer sheath 120.

The resultant alignment of the two shafts ensures that the needle tip 15 is always oriented toward the exit side holes 10 in the outer sheath 120. The exit holes 10 are formed through the section of the wall of the outer sheath 120 where the locking key point 200 is positioned. This design maintains the needle 5 in a pre-determined location while moving between multiple exit holes 10.

When the needle 5 is advanced through the distal end 152 of the inner catheter 110, it must be able to pass through the side exit hole 10. Failure to align the inner catheter 110 and outer sheath 120 will result in the needle tip 15 being unable to access the exit hole 10 and subsequent procedure delays and/or failure. The key hole feature 200 of the outer sheath 120 acts as a guiding channel for the needle distal tip 15 to travel through. The needle 5 centers itself in the key hole 210 because of complimentary geometries of the beveled needle tip 15 and the key hole 210. The needle 5 will thus move along the channel as the inner catheter 110 is withdrawn during tumescent delivery.

The alignment mechanism described herein is only one of various ways to ensure that the inner catheter 110 is oriented correctly and is does not rotate out of position during the procedure. The geometries of the key point 200 and key holes 210 may of different shapes and number. For example, the alignment mechanism could include between one and eight key point/key hole sets. The method of using the single lumen device 100 will now be described, as shown in FIG. 15B. The target vein is accessed using standard technique 433 after which a procedural guidewire is inserted and advanced to the desired location 435. The outer sheath of the device 100 is then inserted over the guidewire 22 and advanced to the desired position within the vessel 437. The guidewire 22 is then removed from the patient 439. The device 100 is then assembled by inserting the inner catheter 110 into the outer sheath 120 by first aligning the inner catheter 441 projecting key points 200 to the outer sheath key holes 210. The longitudinal stripe 24 on the outer surface of the outer sheath 120 may be used as a guide to correctly orienting the inner catheter 110 relative to the sheath 120. The inner catheter 110 is then advanced until the needle tip 15 is positioned at distal most exit side hole. The channel 230 formed by the key points/holes 200/210 ensures that the advancement through the outer sheath 120 does not move out of axial alignment. The ultrasonic visibility of the marker bands 52 and roughened needle tip 15 allow the user to accurately position the needle tip relative to the target tissue surrounding the vein.

The tumescent line 54 extending from the inner catheter handle 155 is attached to a fluid source as previously described 443. Once the needle tip 15 is in the correct position relative to the distal most exit side hole 10 of the sheath 120, the needle 5 is deployed 445. The needle 5 may be deployed through the designated exit hole 10 in several ways. The needle 5 may be advanced distally from the inner catheter shaft 153 directly through the side exit hole 10 into a deployed position using a thumb slide 8 or other deployment mechanism on the inner catheter handle 155. Length of needle deployment into the fascia is controlled by the thumb slide 8. After delivery of fluid 447, as previously described, the needle 5 is retracted completely 449 into the inner catheter 110 and the inner catheter 110 is withdrawn to align the needle tip 15 with the next exit side hole 10 453. The outer sheath 120 is held in a stationary position while the inner catheter 110 is withdrawn to the next exit hole 10. The needle 5 is then redeployed 453 using the markers 52 to identify hole 10 locations. Alternatively, the inner catheter can be advanced to enclose the needle tip 15 and subsequently, the inner catheter 100, with the needle tip 15 can be withdrawn and placed at a proximal location.

Alternatively, the needle 5 may be deployed using a “withdraw and advance” technique. Using the thumb slide, the needle tip 15 is deployed from the inner catheter 110 distally of the designated exit hole 10. The inner catheter 110 is then withdrawn from the outer sheath 120 until the needle tip 15 catches on the proximal side wall of the exit side hole 10. The inner catheter 110 is then advanced slightly, causing the needle tip 15 to “pop” through the exit hole 10 into its unconstrained, curved profile. The thumb slide may be used to adjust needle tip 15 extension into the target tissue. After delivery of fluid, as previously described, the inner catheter 110 is withdrawn with the needle tip 15 remaining in a deployed position. The needle tip 15 tracks along the key point channel 230 of the outer sheath 120. The channel 230 creates less needle tip 15 drag during withdrawal than if the tip 15 was withdrawn while in contact with a non-keyed portion of the sheath inner wall. The key point/hole 200/210 channel 230 also prevents the needle 5 from becoming misaligned relative to the exit holes 10 during the procedure. Once the needle tip 15 passes the next hole 10, the inner catheter 110 is advanced slightly to “pop” the needle tip 15 through the hole 10. Tumescent fluid is injected each time the needle tip 15 is deployed through a hole 10 into the fascia.

Once tumescent fluid has been delivered along the desired length of the vein 455, the needle tip 15 is retracted into the inner catheter lumen 150 and the assembly is removed 457 from the outer sheath 110. The outer sheath 120 which has not been withdrawn during the procedure remains in position within the vessel at the target location. An optical fiber or other delivery device, such as an RF, microwave, or cryo probe, may then be inserted into the outer sheath 110 and advanced until it is positioned at the desired treatment location 457. Thermal energy or other treatment modality, such as the injection of a chemical compound used to close veins, is applied along the entire length of the vein by withdrawing the sheath/fiber assembly at a designated withdrawal rate until the entire vein has been treated 459. This technique is advantageous over prior art methods in that it eliminates steps associated with having to insert a separate thermal ablation procedural sheath after the tumescent delivery step is completed. Instead, the lumen of the outer sheath 120 is designed to accept an optical fiber or other thermal device without any additional procedure steps.

In yet another embodiment (not shown) of the present invention, the device includes two elongated curved needles which when deployed extend through single or multiple corresponding exit side holes. Each needle would “ride” in one of the channels 230 formed by the alignment of key points/key holes 200/210. Alternatively, the device may include a single needle with two or more radially extending, curved branches at the distal section. Each branch deploys through side holes which are positioned 90 to 180 degrees apart at the same longitudinal location on the shaft. Entering the fascial space simultaneously at separate locations may lead to faster and more complete filling of the space without the need to rotate the needle position to ensure 360 degree filling.

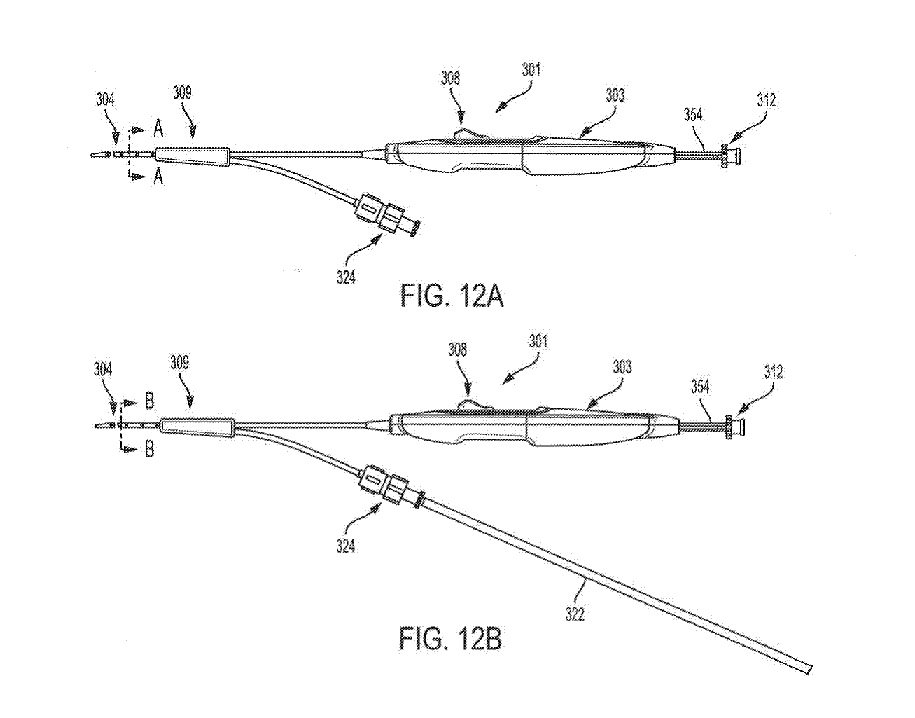

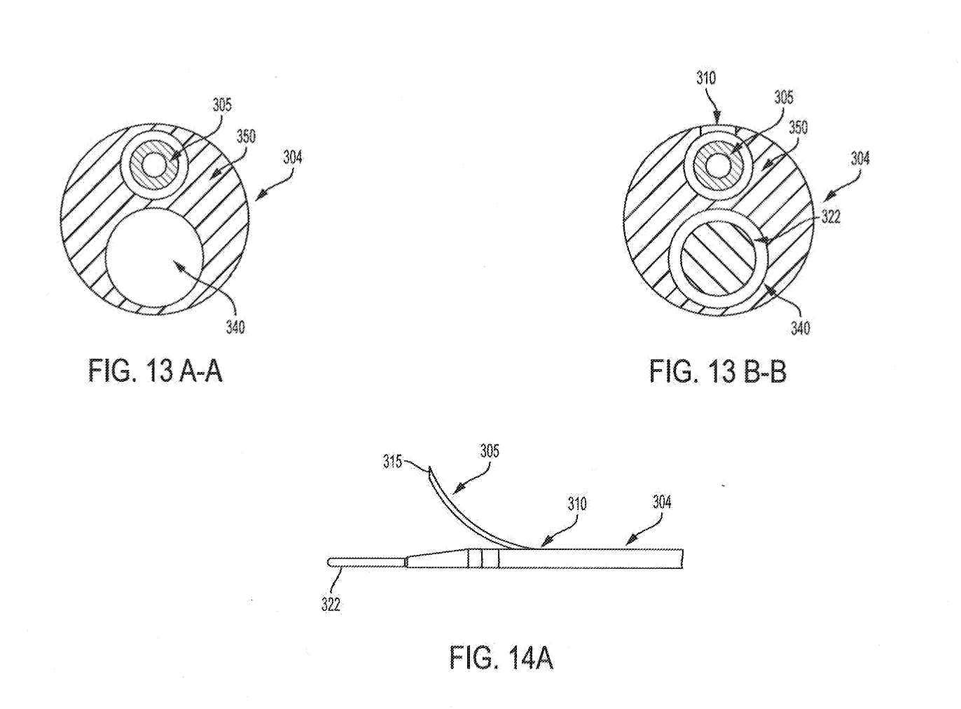

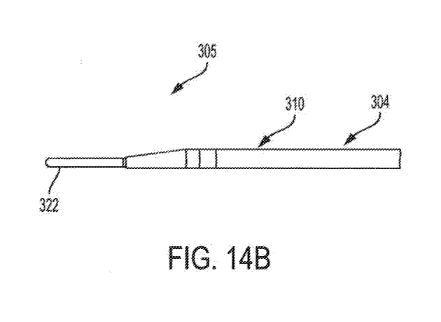

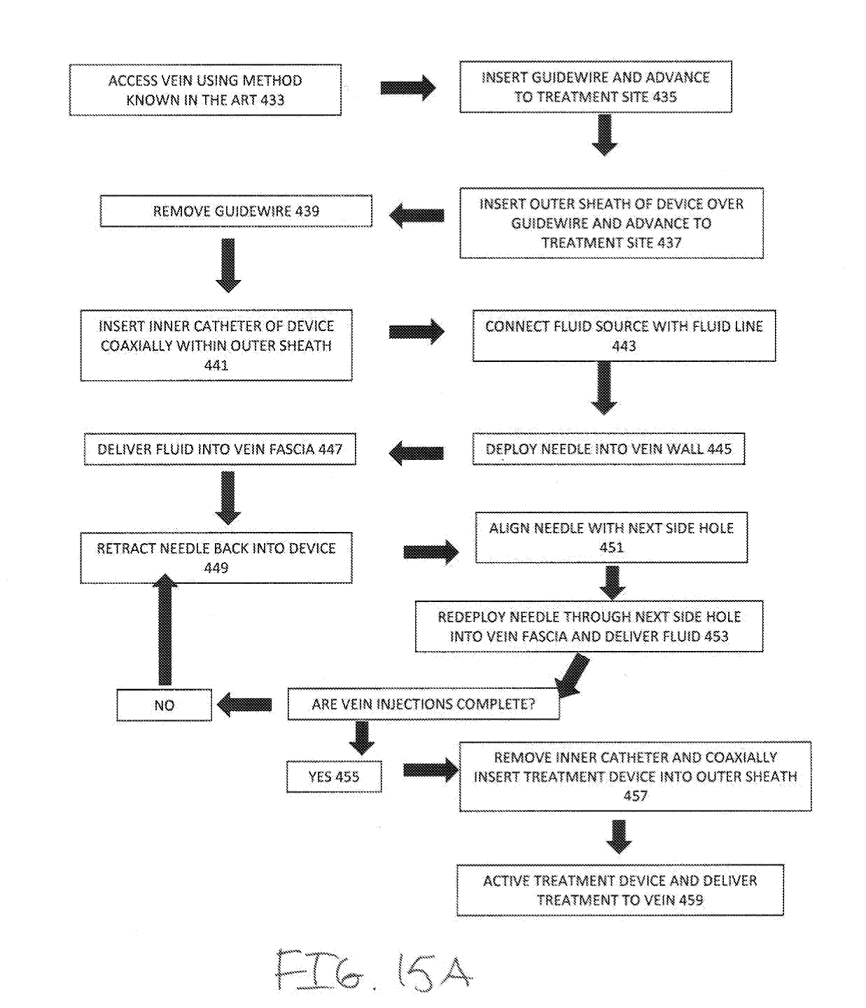

Referring now to FIG. 12A-14B, in yet another embodiment of the present invention includes a device 301 having needle lumen 350 and a energy delivery device lumen 340. The energy delivery device lumen 340 is dimensioned so as to accept either a guidewire during positioning of the device and/or to accept the treatment device 322, which depending on the size of the procedure lumen may be either an optical fiber, an RF probe, or another vein closure modality. The needle 305 is positioned within the needle lumen 350 to provide tumescent injection.

The endoluminal device 301 includes a handle 303 connectable to a tumescent fluid source (not shown) by a fluid source connector 312, a catheter shaft 304 having a double lumen configuration, a hub 309, a device connector 324, a treatment device 322 and a needle 305. In the figures an RF probe is depicted as the treatment device 322; however it is conceived that the bounds of this embodiment the treatment device 322 may also include a laser, cryo device, microwave device or another treatment modality device designed to deliver a chemical agent to treat varicose veins. The handle includes a thumb slide 308 or other mechanism for deployment and retraction of the distal end 315 section of a needle 305 from the catheter shaft 304 through a side exit hole 310. The device connector 324 can be in the form of many connectors in the art, but preferably is a tuohy borst connector. The tuohy borst is preferable in this embodiment because it may allow for less bleedback than a traditional slit valve and will also allow the treatment device 322 to be locked into place for delivering treatment. The double lumen configuration of the catheter shaft 304 comprises a needle lumen 350 and an energy delivery device lumen 340. The shaft size of the catheter shaft 304 can vary in size up to 11F, with a needle lumen with a diameter of up to 0.040″ and a energy delivery device lumen 340 with a diameter of up to 0.092″, although larger diameters are within the scope of this invention depending on the size of the treatment device. The larger diameter of the energy delivery device lumen 340 allows for the use of several larger sized energy delivery devices including, but not limited to a RF probe, a radial fiber, or a larger laser fiber.

The method of using the endoluminal device 301 will now be described and is shown in FIG. 15C. First, the vein is accessed using methods known in the art 461. The device is assembled by first inserting the RF probe or optical fiber through the Tuohy Borst fitting 324. The probe/fiber is then advanced through the fitting 309, through lumen 340 of shaft 304 until the distal end of the ablation fiber/probe extends out of the distal end of shaft 304 by the recommended distance, typically 1-5 cm. Once in the desired position, the Tuohy Borst fitting 324 is tightened down 463 to prevent further movement of the treatment device within the device and to maintain the desired distance between the treatment device distal end and the device distal end. The endoluminal device 301 is then connected to a fluid source 465 by attaching the fluid line 354 of the device 301 to fluid source connector 312. Next, the target vein is accessed using standard technique and a sheath is placed over the guidewire, which is then removed. The sheath size will be dependent on the outer diameter of the endoluminal device 301, which in turn will depend on the size of the ablation device. As an example, for a standard fiber, the sheath may be 8F and for an RF probe 10F.

In this embodiment, the device is inserted into the vein 467 but not advanced to the Saphenous-Femoral Junction. Instead the needle side hole 310 is positioned adjacent to the proximal most tumescent injection site. The needle 305 may then be deployed by moving the thumb slide 308 distally. The movement of the thumb slide 308 distally, causes the distal end section 315 of needle 305 to exit the catheter shaft 304 through the side exit hole 310, as shown in FIG. 6 and pierce the wall of the vein, ending up in the fascia of the vein 469. Once the distal end section 315 of needle 305 has pierced the wall of the vein, tumescent or any other fluid is delivered through the needle 305 into the fascia of the vein 471. Once the tumescent or other fluid has been delivered to the fascia of the vein, the needle 305 is retracted back 473 through the side exit hole 310 and into the needle lumen 350 by moving the thumb slide 308 proximally. After the needle 305 has been retracted back into the needle lumen 350, the endoluminal device 301 is advanced distally to a new position within the vein and tumescent is delivered again using the steps described above 475. These steps are repeated until tumescent fluid has been delivered to the entire treatment length of the vein 477 and the distal end of the ablation device is positioned near the Sapheous-Femoral junction or other desired location.

The user then activates the RF probe/fiber 322. Activation and use of an RF probe is known in the prior art and is described in U.S. Pat. No. 6,769,433, filed May 25, 2001. Laser ablation procedures are also known in the prior art as described in U.S. Pat. No. 7,559,329, which is herein incorporated by reference. Once the probe 322 is activated, the user can withdraw the endoluminal device 301 at a designated withdrawal rate until the entire vein has been thermally treated 479.

Varicose vein treatment using the above described embodiment has several advantages. The advancement of the endoluminal device 301 with all of the necessary components assembled requires for less steps in the treatment process, which results in a much quicker treatment of the vein and less discomfort for the patient. This ability to deliver tumescent as the endoluminal device 301 is being inserted and advanced into the vein is an advantage because the RF probe 322 is already attached to the endoluminal device 301, negating the use of a guidewire and the need to remove the endoluminal device 301 after delivering tumescent in order to insert the fiber/probe to the endoluminal device 301 to deliver treatment. In addition to requiring less component swaps, the method herein also is advantageous in that the number of components required to perform the procedure is decreased, thus reducing overall costs.

Another embodiment (not shown) of the present invention includes a device having three lumens, including a guidewire lumen, a needle lumen and a treatment device lumen. The method of using such a device may include inserting the guidewire through the guidewire lumen and into the vessel and advancing to the target site. The treatment device is then inserted into the treatment device lumen and then the entire device is advanced over the guidewire into position at the treatment site. The needle may be preloaded into the device. Tumescent fluid is administered using multiple side holes in the device while the device remains stationary within the vessel, as was described above. Then with the treatment device already positioned at the starting treatment point, the vessel is treated as the entire device is slowly pulled back in a proximal direction. This method is advantageous in that the device is only advanced once during the procedure without any component exchanges required.

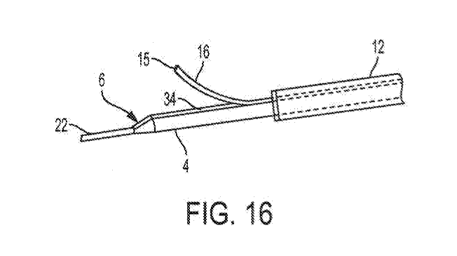

Another embodiment of the current invention is shown in FIG. 16 and includes an endoluminal device where instead of a dedicated needle lumen within the device, the device has a needle track 6 disposed on the catheter. The needle of the device is made of nitinol or some memory shape alloy and has a distal tip 15 that, when not deployed, rests within the needle track 6. The needle is kept in the needle track by a cover sheath 12 that is fed over the catheter shaft 4 of the endoluminal device. When the cover sheath 12 is advanced over the catheter shaft 4, the needle is held in place in the needle track 6, but when the cover sheath 12 is withdrawn, the needle deploys.

The treatment device may include, but not limited to, a device capable of delivering laser energy, steam, RF, plasma, cryotherapy and microwave among others. The embodiments describe herein may also be adapted for use with sclerotherapy or other fluid delivery within the vein or other target structure. As an example, an occluding ball wire, such as is described in U.S. Pat. No. 6,283,950, which is incorporated herein by reference, may be used to occlude the end hole of the catheter or outer sheath. Sclerosant fluid or foam injected into the catheter lumen would exit through the side holes to treat the vein. Alternatively, the laser fiber distal end section could be used to occlude the distal end of the endoluminal treatment device as described in U.S. patent application Ser. No. 11/303,818.

The endoluminal device described herein may also be used as a delivery system to administer localized anesthesia or other medicinal fluids to anatomical target areas not easily accessible via a percutaneous approach. The device could be advanced through other veins, larger arteries, ducts and other anatomical tubular structures for the delivery of fluid through the wall of the tubular structure to an adjacent target area. This design would allow the clinician to bypass sensitive tissue while knowing the exact location of the needle tip to ensure accurate and precise delivery of the medicinal fluid. The present invention can be understood more readily by reference to the following detailed description, the examples included therein, and to the Figures and their following description. The drawings, which are not necessarily to scale, depict selected preferred embodiments and are not intended to limit the scope of the invention. The detailed description illustrates by way of example, not by way of limitation, the principles of the invention. The skilled artisan will readily appreciate that the devices and methods described herein are merely examples and that variations can be made without departing from the spirit and scope of the invention. It is also to be understood that the terminology used herein is for the purpose of describing particular embodiments only and is not intended to be limiting.

What is claimed is:

1. A method of delivering a fluid to a vessel comprising:
inserting a guide wire into the vessel;
inserting the proximal end of said guide wire through an endoluminal device, the endoluminal device having a proximal end, a distal end, and at least one lumen, the endoluminal device having at least one hole along an outer wall;
advancing a needle through the lumen;
advancing the needle through one of the holes and thereby piercing the vessel wall with the needle;
delivering the fluid to the vessel;
retracting the needle in a proximal direction until the needle is situated at the next most proximal hole;
advancing the needle through the next most proximate hole;
removing the needle from the endoluminal device;
advancing a treatment de ice through the endoluminal device to a selected position distal to the distal most end of the endoluminal device.

2. The method of claim 1, further comprising, after advancing the needle through the next most proximate hole, the user then repeats the steps of advancing the needle through a hole, delivering the fluid to the vessel by piercing the vessel wall, and retracting the advanced needle to another location before delivering the medicinal substance to the next location.

3. The method of claim 2, further comprising after the step of advancing a treatment device through the endoluminal device to a selected position distal to the distal most end of the endoluminal device, longitudinally moving the endoluminal device together with the treatment device to a second position to ablate the vessel.

4. The method of claim 1, wherein the at least one lumen includes a first lumen and a second lumen in communication with the hole.

5. The method of claim 1, wherein at least part of the needle is further comprised of a pre-shaped memory material.

6. The method of claim 1, wherein the endoluminal device further comprises a handpiece assembly is coupled to the proximal end of the endoluminal device, the handpiece assembly including a button coupled to the needle; and advancing the needle through the hole includes pressing the button.

7. The method of claim 1, wherein the endoluminal device further comprises a fluid barrier layer.

8. The method of claim 7, further comprising flushing the device with fluid prior to advancing the needle so as to keep fluid from one or more of the plurality of holes along the outer wall.

9. The method of claim 7, wherein the step of advancing the needle includes the needle piercing the fluid barrier layer prior to delivering fluid to the vessel.

10. An endoluminal device comprising;
a shaft comprising a proximal end, a distal end, an outer sheath, an inner sheath, and a longitudinal axis;
the inner and outer sheath each having a keyed geometry;
the inner shaft having a needle inside the lumen; and
the outer shaft having a channel for guidance of a needle.

11. The device of claim 10, wherein the keyed geometry of both the inner and outer sheath allow the two to be slidably interacted with one another.

12. The device of claim 10, further comprising a plurality of holes along the longitudinal axis of the outer shaft.

13. The device of claim 10, further comprising a fluid barrier layer covering at least one side hole in the outer shaft.

14. The device of claim 12, wherein a marker band is placed on the distal portion of each of the plurality of holes along the longitudinal axis of the outer shaft.

15. The device of claim 10, further comprising a handpiece assembly connected to the proximal end of the inner shaft.

16. The device of claim 13, wherein the fluid barrier layer covers substantially all of the side holes.

17. The device of claim 16, wherein the button is connected to the needle of the inner shaft.

18. The device of claim 16, wherein the needle of the inner shaft is deployed by pressing the button.

19. A method of delivering a fluid to a vessel comprising:
accessing a target vein of a patient to receive treatment;
preparing an endoluminal device to be inserted into the accessed vein, the endoluminal device having a proximal end, a distal end, and a catheter shaft; the catheter shaft further comprising a needle lumen, a treatment device lumen; the endoluminal device having at least one exit hole along an outer wall of the catheter shaft;
inserting the endoluminal device into the vein over a guidewire;
removing the guidewire;
inserting the treatment device into the treatment device lumen until the treatment device extends a selected distance distally beyond a distal most end of the endoluminal device;
securing the treatment device with the endoluminal device;
advancing the endoluminal device to a first injection site;
deploying a needle through the needle lumen into a vessel wall;
delivering the fluid to the vessel through the needle;
advancing the endoluminal device distally to a second injection site;
delivering the fluid to the at the second injection site vessel through the needle;
advancing the endoluminal device to a treatment site;
activating the treatment device;
withdrawing the endoluminal device.
